(19) United States
(12) Patent Application Publication
(10) Pub. No.: US 2007/0196488 A1
(43) Pub. Date: Aug. 23, 2007

(54) Oral matrix formulations comprising licarbazepine

(76) Inventors: Oskar Kalb, Lörrach (DE); Marie-Christine Wolf, St. Louis (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/598,786

(22) PCT Filed: Mar. 21, 2005
(86) PCT No.: PCT/EP05/02988

§ 371 Date: Sep. 12, 2006

(30) Foreign Application Priority Data
Mar. 22, 2004 (GB) 0406379.8
Mar. 25, 2004 (GB) 0406738.5

(51) Int. Cl. A61K 31/47

(52) U.S. Cl. 424/474; 514/217

(57) ABSTRACT

The invention relates to pharmaceutical compositions comprising 10,11-dihydro-10-hydroxy-5H-dibenz[b,f]azepine-5-carboxamide (also referred to as “licarbazepine”) as drug substance.

The present invention relates to pharmaceutical compositions comprising 10,11-dihydro-10-hydroxy-5H-dibenz[b,f]azepine-5-carboxamide (also referred to herein as “licarbazepine”) as drug substance.

The term licarbazepine as used herein refers to the racemic mixture of (S)-10,11-dihydro-10-hydroxy-5H-dibenz[b,f]azepine-5-carboxamide and (R)-10,11-dihydro-10-hydroxy-5H-dibenz[b,f]azepine-5-carboxamide.

In the present invention licarbazepine, mixtures of (S)-10,11-dihydro-10-hydroxy-5H-dibenz[b,f]azepine-5-carboxamide and (R)-10,11-dihydro-10-hydroxy-5H-dibenz[b,f]azepine-5-carboxamide comprising one of the two enantiomers in excess, or one of the essentially pure or pure enantiomers of licarbazepine can be employed as drug substance and are all together hereinafter referred to as the “compounds of the invention”.

Licarbazepine (also known as MHD) is well known from the literature [see, for example, Schuetz H. et al., Xenobiotica (GB), 16(8), 769-778 (1986)] and can be prepared synthetically, for example starting from oxcarbazepine, according to conventional methods, e. g. as described in U.S. Pat. No. 3,637,661.

The pure enantiomers of licarbazepine can be obtained starting from the racemate by procedures known as such. For instance, the racemate may be separated into its enantiomers through the formation of diastereomers, e. g. as disclosed in WO-02/092572, or, alternatively, by salt formation with an enantiomer-pure chiral acid, or by means of chromatography, for example by HPLC, using chromatographic substrates with chiral ligands. In one embodiment of the invention, the pure enantiomers of licarbazepine are prepared by an enantioselective process described in the Examples.

Licarbazepine is indicated to be suitable for the treatment of psychosomatic disturbances, epilepsy, trigeminal neuralgia and cerebral spasticity. It was demonstrated that the racemate of licarbazepine and both of its pure enantiomers are of equal efficacy against epilepsy. The mechanisms by which the compounds of the invention exert their anticonvulsant effects are not completely understood, but their activity may be partly due to effects on ion flow across neuronal membranes. However, pharmacokinetics, absorption sites and mechanisms of action of the compounds of the invention are not understood in detail.

Licarbazepine is slightly soluble in water (3.2 mg/ml at 25° C.). In view of this physical property, a parenteral formulation of licarbazepine can be prepared as described, e. g., in EP-1033 988. Despite the merits of the known parenteral dosage form, there remains a need to establish an advantageous oral dosage form of the compounds of the invention. One of the problems that may occur using an oral dosage form is the fluctuation of blood levels of the compounds of the invention on repeated administration, which may be associated with side effects.

After exhaustive testing, advantageous pharmaceutical oral controlled release compositions, which are capable of being administered once a day and which are particularly well tolerated and have a good bioavailability in a wide variety of patient populations, have now surprisingly been found.

Hence, in one aspect, the present invention relates to pharmaceutical oral controlled release compositions adapted to be administered once a day comprising at least one of the compounds of the invention (hereinafter referred to as “oral dosage forms of the invention”), in particular showing a low fluctuation index for a better tolerability and a continuous symptom control with an adequate Cₘᵢₙ (Minimum Plasma Concentration) value and furthermore having the advantage of a high AUC (Area Under the Curve) and a low Cₘₐₓ (Maximum Plasma Concentration) value.

The oral dosage forms of the invention may represent a considerable advantage over other oral dosage forms in that they are more convenient and/or safer for patients to use and increase the patients' compliance to therapy. The patients have to take the oral dosage forms of the invention only once a day.

The term “once a day” as used herein means once every 20 to 28 hours, in particular once every 24 hours.

Preferred oral dosage forms of the invention comprise the compounds of the invention, especially licarbazepine, and a lipophilic or hydrophilic, preferably hydrophilic, swellable substance.

In such oral dosage forms, the compounds of the invention, especially licarbazepine, can be present in an amount of from 55 to 80%, preferably from 65 to 70%, e. g. in an amount of about 68%, by weight of the total composition.

The compounds of the invention, especially licarbazepine, are preferably employed in fine form, i. e. having a median particle size (x₅₀) of from about 20 to about 50 μm, preferably from about 30 to about 50 μm, more preferably from about 35 to about 45 μm, e. g. of about 40 μm.

Swellable substances commonly used in tablet formulations may be used, and reference is made to the extensive literature on suitable swellable substances, in particular to Fiedler's “Lexikon der Hilfsstoffe”, 4th edition, ECV Aulendorf (1996), hereinafter referred to as “LdH”, and to “Handbook of Pharmaceutical Excipients”, Wade and Weller, 3rd ed. (2000), hereinafter referred to as “HoPE”, which are incorporated herein by reference.

In one embodiment of the invention, the oral dosage form comprises at least one hydrophilic swellable substance selected from the group of compounds, consisting of natural, or partially or totally synthetic, hydrophilic gums, cellulose derivatives and protein aqueous substances, preferably consisting of natural, or partially or totally synthetic, anionic or, preferably, nonionic, hydrophilic gums, modified cellulose substances and protein aqueous substances, for example consisting of acacia, gum tragacanth, locust bean gum, guar gum, karaya gum, agar, peptin, carrageen, soluble or insoluble alginates, methyl cellulose, hydroxypropyl methyl cellulose, hydroxypropyl cellulose, hydroxyethyl cellulose, sodium carboxymethyl cellulose, carboxypolymethylene and gelatin, preferably consisting of cellulose substances, such as methyl cellulose, hydroxypropyl cellulose and hydroxypropyl methyl cellulose. Especially preferred is hydroxypropyl methyl cellulose.

The swellable substances employed according to the invention with diverse viscosities may be prepared as disclosed in HoPE.

The swellable substance can be present in an amount of from about 5 to about 45%, preferably from about 5 to about 35%, more preferably from about 10 to about 15%, e. g. in an amount of about 13%, by weight of the total composition.

The weight ratio of the swellable substance to the drug substance, especially licarbazepine, may be from about 1:3 to about 1:10, preferably from about 1:4 to about 1:7, more preferably from about 1:5 to about 1:6.

The swellable substance can be a mixture of 2 or more than 2 swellable substances.

In a further aspect, the present invention relates to pharmaceutical oral controlled release compositions comprising licarbazepine, characterized in that in use from about 70 to about 90%, preferably from about 80 to about 90%, of said licarbazepine are released within from about 8 to about 12 hours, indicated in standard in-vitro dissolution tests at 37° C. in aqueous phosphate buffer preferably having a pH of about 6.8 for a 500 mg dosage form, e. g. effected using the apparatus 2 (Rotating Paddle) of the USP at a stirring rate of 50 rpm (hereinafter referred to as “in-vitro licarbazepine dissolution test conditions of the invention”).

In one embodiment of the invention, the oral dosage form comprises a tablet core and a coating, the tablet core comprising the drug substance, especially licarbazepine, at least one hydrophilic swellable cellulose ether, preferably hydroxypropyl methyl cellulose, and, optionally, a filler. The weight ratio of the hydroxypropyl methyl cellulose to the drug substance, especially licarbazepine, in such an oral dosage form may be preferably from about 13 to about 1:10, preferably from about 1:4 to about 1:7, more preferably from about 15 to about 1:6.

Clinical studies, for instance bioavailability trials, may be effected in a conventional manner. For example, they may be effected over 7 or more days using a 500 mg dose of a compound of the invention. Conveniently at least 6, e. g 10, subjects are enrolled. In such studies modified release characteristics, bioavailability, food effect, safety, tolerability, Cₘₐₓ, Cₘᵢₙ and/or AUC of the oral dosage forms of the invention can be determined.

The bioavailability of a drug substance depends on its physicochemical properties, such as solubility, and pharmacokinetic properties, e. g. site, rate and extent of absorption. Further, it is known, that food induces changes in the physiology of the gastrointestinal (GI) tract. These changes can result inter alia in delays in gastric emptying, stimulation of bile flow and changes in pH. Food can also alter lumenal metabolism and physically or chemically interact with a drug substance. It is not surprising, therefore, that food can also effect the bioavailability of a drug substance. The term “food effect” as used herein means, that the bioavailability of a drug substance in a subject in the fed state differs from the bioavailability of this drug substance in a subject in the fasted state. The effects of food are complicated and difficult to predict and will depend, for example, on the nature of the meal, e. g. its nutrient content, fluid volume, caloric content and temperature. It follows, that the presence or absence of a food effect for a given drug substance can only be determined after exhaustive testing.

It is undesirable, if the bioavailability of a drug substance differs depending upon whether a patient is in a fed or fasted state. This will at least be inconvenient to the patient, who will have to time its medication relative to the taking of meals.

It is surprising, therefore, that it was discovered that an oral dosage form of licarbazepine may be administered to a patient without regard to the condition of the patient, i.e. whether the patient is in a fed or fasted state.

Accordingly, the present invention relates in a further aspect to an oral dosage form of the invention having no food effect when administered to a patient.

In a further aspect, the present invention relates to a package comprising an oral dosage form of the invention and, e. g. written, instructions for use, said instructions providing that the oral dosage form may be taken equally by patients who have eaten or who are in a fasted condition.

More particularly, the present invention relates to an oral dosage form of the invention packaged in combination with, e. g. written, instructions, which instructions provide that the oral dosage form may be taken equally with or without food.

The presence or absence of a food effect may be quantified by making AUC measurements and/or Cₘₐₓ measurements according to methods well known in the art. Typically, such measurements are made by taking timed biological fluid samples and plotting the serum concentration of the drug substance, e. g. licarbazepine, against time. The values obtained represent a number of values taken from subjects across a patient population and are, therefore, expressed as mean values over the entire patient population. By comparing the mean AUC and/or Cₘₐₓ values, one can determine whether the drug substance, e. g. licarbazepine, exhibits a food effect.

A “fed” subject conveniently may be considered as a subject, that has fasted for at least 10 hours before having received a standard FDA recognised high fat meal. The drug substance, e. g. licarbazepine, may then be administered with water shortly after completion of the meal, e. g. within 5 minutes thereof. Preferably no food should be taken for a period of, e. g., 4 hours after administration of the drug substance, e. g. licarbazepine, although small quantities of water may be permitted after, e. g., 2 hours after administration of the drug substance, e. g. licarbazepine.

A “fasted” subject conveniently may receive the drug substance, e. g. licarbazepine, with water after at least 10 hours of fasting. Thereafter, no food may be taken for a period of, e. g., 4 hours, although small quantities of water may be taken after, e. g., 2 hours after administration of the drug substance, e. g. licarbazepine.

A “standard FDA recognised high fat meal” as referred to herein may comprise any meal, that would be expected to provide maximum perturbation due to the presence of food in the GI tract. Said high fat meal typically may comprise 50% of its caloric value in fat. A representative example may be 2 eggs fried in butter, 2 stripes of bacon, 2 slices of toast with butter, 4 ounces of fried potatoes and 8 ounces of milk.

To study the effect of food on the bioavailability of a drug substance one may use any conventional study design known in the art, for example a randomised, balanced single-dose, two-treatments, two-periods, two-sequences, crossover design. The analysis may be carried out using software from the SAS institute, Cary, N.C., e. g. SAS PROC GLM.

A suitable study design to determine the bioavailability, including the food effect, of an oral dosage form of the invention would be a randomized, open-label, single oral dose, crossover study, wherein one can compare the bioavailability of the oral dosage form of the invention comprising a compound of the invention with the bioavailability of a solution of the same compound of the invention, optionally also including oxcarbazepine film coated tablets, and evaluate the food effect in healthy male subjects being in a fed or fasted state.

In a study, wherein the drug substance is, for instance, licarbazepine, the oxcarbazepine film coated tablet (600 mg) and the oral dosage form of the invention comprising, e. g., 500 mg of licarbazepine can be administered together with 240 ml of tap water to the subjects. The licarbazepine clinical service form (500 mg) delivered as powder has to be solubilized in the tap water prior to the drug administration. During the treatment periods that require fasted conditions, the single dose of the study drugs is administered after an overnight fast of at least 10 hours. During the treatment periods that require fed conditions, each subject is requested to eat a standard FDA recognised high fat breakfast within 30 minutes prior to the drug administration. No breakfast is served prior to the drug administration during the treatment periods that require fasted conditions, and the subjects have to continue to fast until 4 hours postdose. The safety and tolerability monitoring includes continuous monitoring of adverse events, physical examinations, blood pressure and pulse rate measurements, ECG recordings and routine laboratory tests (blood chemistry, urinalysis and hematology).

During a first 7 days period, the subjects will be given one of the oral dosage forms of the invention under fasted conditions, and during the second period the subjects will be given the same treatment under fed conditions. The subjects will fast overnight for a minimum of 10 hours on the evening prior to the first dosing of a compound of the invention (period 1). Following dosing at. e. g, breakfast time, pharmacokinetic blood samples may be drawn and used for assays at adequate time intervals, e. g. 0.5, 1, 2, 3, 4, 6, 8, 10, 12, 14, 16, 18, 20, 22, 24, 32 and 48 hours after administration.

The absorption profile of the compound of the invention may be quantified by making AUC measurements on single doses or at the steady state.

Constant plasma levels of the compound of the invention indicate, that the plasma levels of the compound of the invention show low fluctuation indices. The Cₘᵢₙ and Cₘₐₓ values of the compound of the invention may be kept within a small range. To measure the fluctuation between Cₘᵢₙ and Cₘₐₓ, the compound of the invention plasma levels are measured at the steady state, and the fluctuation index is calculated according to (Cₘₐₓ−Cₘᵢₙ)/Cₐᵥ (wherein Cₘₐₓ is the maximum concentration, Cₘᵢₙ is the minimum concentration and Cₐᵥ is the average concentration, observed within a certain time interval, e. g. 24 hours, at the steady state).

The low fluctuation of Cₘᵢₙ and Cₘₐₓ may avoid peak values of the compound of the invention plasma levels, which can be toxic for the patient. A lower fluctuation may provide better tolerability and safety for the patient treated with a compound of the invention.

Accordingly, in a further aspect, the present invention relates to a method of reducing the intra-subject variability of the bioavailability levels of licarbazepine in a patient during oral licarbazepine therapy, said method comprising administering an oral dosage form of the invention comprising licarbazepine as drug substance, which shows no food effect when administered to such patient indiscriminately in the fed or fasted state, e. g. at any hour.

In a further aspect, the present invention relates to the use of licarbazepine for the preparation of a medicament for the treatment of patients with affective disorders.

The term “affective disorders” as used herein includes, but is not limited to, uni- and bipolar depression, bipolar disorder, pre-menstrual dysphoric disorder, post-partum depression, post-menopausal depression, neurodegeneration-related depressive symptoms, depression occurring following cessation of psychostimulant intake, psychotic states, e. g. mania, schizophrenia and excessive mood swings where behavioural stabilization is desired.

The utility of the oral dosage forms of the invention for the treatment of affective disorders may be observed in standard animal tests or in standard clinical studies, for example in clinical studies in bipolar disorder patients, with administration of, for example, dosages of licarbazepine in the range of from about 500 to about 3000 mg per day.

The oral dosage forms of the invention may be produced in conventional manner by mixing the components. The resultant mixture may be in powder form, which may be pressed to form a tablet in conventional tabletting machines.

Conveniently oral dosage forms of the invention may be produced by compressing a compound of the invention with e.g. conventional tabletting excipients to form a tablet core using conventional tabletting processes and subsequently coating the core. The tablet cores can be produced using conventional granulation methods, for example wet or dry granulation, with subsequent compression and coating. Granulation methods are described, for example, in R. Voigt, Lehrbuch der Pharmazeutischen Technologie, Verlag Chemie, 6^{th} edition, pages 156-169.

Granules may be produced in a manner known per se, for example using wet granulation methods known for the production of “built-up” granules or “broken-down” granules.

Methods for the formation of built-up granules may comprise, for example simultaneously spraying the granulation mass with granulation solution and drying, for example in a drum granulator, in pan granulators, on disc granulators, in a fluidised bed, by spray-drying or spray-solidifying, or operate discontinuously, for example in a fluidised bed, in a batch mixer or in a spray-drying drum.

Depending on the method used, the granulation mass may be in the form of a premix or e.g. may be obtained by mixing a compound of the invention with one or more excipients. The wet granules are preferably dried, for example by tray drying or in a fluidised bed.

Oral dosage forms of the invention may contain, in addition to a compound of the invention, conventional excipients depending on the exact nature of the formulation. Suitable categories of excipients include fillers, lubricants, film coating agents, binders, glidants, solubilizers, surface-active substances and disintegrants.

Excipients disclosed in the literature, as for instance in Fiedler's “Lexikon der Hilfstoffe”, 4^{th} Edition, ECV Aulendorf and “Handbook of Pharmaceutical Excipients”, Wade and Weller, Third Edition (2000), the contents of which are incorporated herein by reference, may be used in the pharmaceutical compositions according to the invention. Conveniently the excipients comprise less than 40% of the weight of the dosage form.

We have found that certain excipients exhibit especially interesting properties in oral dosage forms of the invention comprising licarbazepine, e.g. cellulose ethers, such as

i) hydroxypropyl methylcellulose, e.g.
Methocel 60 HG 4000 CP, preferably in a weight ratio of from about 1:4 to about 1:8, Methocel HG which has a 2 percent aqueous viscosity of approximately 4000 mPa s, a methoxyl content of 26 to 30%, and a hydroxypropyl content of 7 to 12%.
CR grade Methocel E-4M, which has a 2 percent aqueous viscosity of approximately 4,000 mPa s, a number average molecular weight of approximately 90,000, a methoxyl content of 28.0 to 30.0%, and a hydroxypropoxyl content of 7.0 to 12.0% or equivalent, e.g. 10-20% by tablet.
Methocel E-50 Premium, which has a 2 percent aqueous viscosity of approximately 50 mPa s, a number average molecular weight of approximately 20,000, a methoxyl content of 28.0 to 30.0%, and a hydroxypropoxyl content of 7.0 to 12.0% or equivalent (e.g. 10-20% by weight per tablet).
A preferred weight ratio of total hydroxypropylmethyl cellulose to licarbazepine is from about 1:3 to about 1:10 within the granulate and within the external phase of the granulate.
Hydroxypropyl methyl cellulose (HPMC) polymers may be used as matrix components modifying the release of the drug, either alone or in combination with other materials. Oral dosage forms of the invention containing HPMC polymers may prolong drug release by forming a swelling matrix upon exposure to the aqueous medium of the stomach which prevents or delays ingress of the aqueous medium of the stomach into the dosage form and thereby preventing its rapid disintegration. The gel matrix may be formed as a result of hydration of the HPMC polymer. Insignificant instability problems during storage of the oral dosage form of the present invention comprising oxcarbazepine, excipients in combination with HPMC may occur.
A preferred excipient to use as a matrix component is a cellulose ether product such as methylcellulose and hypromellose. Such hypromellose products may be made wherein propylene oxide is used in addition to methyl chloride to obtain hydroxypropyl substitution on the anhydroglucose units in cellulose. This substituent group, —OCH2CH(OH)—CH3, contains a secondary hydroxyl on the number two carbon and may also be considered to form a propylene glycol ether of cellulose. These products possess varying ratios of hydroxypropyl and methyl substitution, a factor which influences organic solubility and the thermal gelation temperature of aqueous solutions.
Viscosities are preferably from 100 to 120.000 mPas at 20° C.
Such products include Methocel products available from Dow Chemical company USA. An alternative is an ethyl cellulose such as Aquacoat® available as a 30 wt. % ethylcellulose dispersion from FMC or Surelease.
Hydroxypropylmethyl cellulose (as mentioned above) is a preferred excipient, for example the quality of Cellulose HPM 603 which has a viscosity of about 3 mPa s, available e.g. as Pharmacoat® 603 (Fiedler, loc.cit., p. 1172). It may act as a binder. Cellulose derivatives such as hydroxypropylmethylcellulose, preferably have a molecular weight of from 10 000 to 1500 000 Daltons.

ii) Ethylcellulose, e.g. Ethocel Premium 7 cps, which has a 2 percent aqueous viscosity of approximately 7 cps and an ethoxyl content of 44.0 to 51.0% or equivalent e.g. 7-10%.

iii) Hydroxypropylcellulose, e.g. Klucel LF, which has a 5% viscosity of approximately 100 cps, and a hydroxypropoxyl content of approximately 54 to 77% or equivalent (e.g. 0.5-5% by weight per tablet) or hydroxyethyl cellulose (HEC).

Hydroxypropyl cellulose may be e.g. hydroxypropyl cellulose having a hydroxypropyl content of 5 to 16% by weight and a molecular weight of from 80,000 to 1,150,000, more particularly 140,000 to 850,000

Examples of other binders include
starches, e.g., potato starch, wheat starch, corn starch; e.g. having a molecular weight of from 30 000 to 120 000,
polyvinyl pyrrolidone, e.g., Povidone, especially having a mean molecular weight of approximately 1000 and a degree of polymerisation of approximately from 500 to 2500, and polymethylacrylates, having a molecular weight of >100 000 Daltons, for example copolymers of acrylic or
methacrylic acid esters, known as Eudragit RL 30D (Handbook of Pharmaceutical Excipients loc.cit., p. 402).

Microcrystalline cellulose is preferably present. It may be used as a filler. Examples include the Avicel® type (FMC Corp.), for example of the types AVICEL PH101, 102, 105, RC581 or RC 591 (Fiedler loc.cit., p. 216), Emcocel® type (Mendell Corp.) Elcema® type (Degussa), Filtrak® type, Heweten® type or Pharmacel®. Preferably the weight ratio of microcrystalline cellulose to the compound of the invention is from about 1:3 to about 1:6, more preferably 1:4 to 1:5.

Another preferred filler is for example a pulverulent filler especially optionally having flow-conditioning properties, including carbohydrates, such as sugars, sugar alcohols, starches or starch derivatives, for example lactose, dextrose, saccharose, glucose, sorbitol, mannitol, xylitol, potato starch, maize starch, rice starch, wheat starch or amylopectin, tricalcium phosphate or calcium hydrogen phosphate.

Polyvinyl-polypyrrolidone is preferably present. Conveniently it functions as a disintegrant. A preferred example is a crosslinked polyvinylpyrrolidone, e.g. crospovidones, e.g. Polyplasdone® XL (Fiedler loc.cit., p. 1245) and Kollidon® CL disintegrant.

Examples of other disintegrants include: (i) natural starches, such as maize starch, potato starch, and the like, directly compressible starches, e.g. Sta-rx® 1500, modified starches, e.g. carboxymethyl starches and sodium starch glycolate, available as Primojel®, Explotab®, Explosol®, and starch derivatives such as amylose; (ii); crosslinked sodium carboxymethylcellulose, available as e.g. Ac-di-sol®, Primellose®, Pharmacel® XL, Explocel®, and Nymcel® ZSX; (iii) alginic acid and sodium alginate; (iv) methacrylic acid-divinylbenzene copolymer salts, e.g. Amberlite® IRP-88, and vi).magnesium aluminium silicate, bentonite, alginic acid and alginates.

Colloidal silicas e.g. Aerosil 200 (Fiedler, loc.cit., p 117) may be preferably present. These may act as a glidant. Examples of other glidants include: silica, magnesium trisilicate, powdered cellulose, starch, talc and tribasic calcium phosphate.

Magnesium stearate is a preferred excipient. It may function as a lubricant. Examples of other lubricants include: calcium stearate, zinc stearate, talc, polyethylene glycol, stearic acid, sodium benzoate, sodium dodecyl sulfate, also know as sulphuric acid monododecyl ester sodium salt, mineral oil, and polyoxyethylene monostearate. A combination of lubricants may also be used.

A granulate of licarbazepine may be coated. Suitable coating materials include those materials conventionally used in coating tablets, granules and the like. In one group of embodiments the coating is water soluble. In another group of embodiments the coating is gastric juice resistant but soluble in intestinal juices.

Unless otherwise indicated, all percentages are weight by weight.

Oral dosage forms of the invention may be combined with immediate release systems. A combination may be a double-layer tablet comprising an immediate release system and a matrix system wherein a compound of the invention, e.g. licarbazepine. A double-layer tablet may comprise two doses of a compound of the invention, one part being adapted to provide a sustained release dose and another part adapted to provide an immediate release dose. For tablets comprising licarbazepine, by immediate release is meant release of at least 90% of the dose within 0.5 hours and 100% of the dose within 1.5 hours under in vitro licarbazepine test dissolution conditions of the invention.

In one embodiment of the invention, preferably a 500 mg licarbazepine dose is used.

Furthermore, the invention provides
a pharmaceutical oral controlled release composition comprising 10,11-dihydro-10-hydroxy-5H-dibenz[b,f]azepine-5-carboxamide and a hydrophilic swellable substance adapted to be administered once a day;
a method of orally administering 10-hydroxy-10,11-dihydrocarbamazepine, e.g., for the treatment of affective disorders, said method comprising orally administering to a patient in need of 10-hydroxy-10,11-dihydrocarbamazepine therapy once-a-day a pharmaceutical composition according to any one of claims 1 to 11.

Following is a description by way of example only of compositions and processes of the invention. In Example 1, 500 mg of drug substance (fine drug substance; X₅₀: 40 microns) is employed. In a similar manner tablets can be prepared comprising 750 mg, 250 mg or 125 mg of drug substance.

Abbreviations

Ac acetyl

aqu. aqueous

dansyl 5-(dimethylamino)-1-naphthalenesulfonyl

Et ethyl

HPLC high pressure liquid chromatography

Me methyl

NMR nuclear magnetic resonance

RT room temperature

THF tetrahydrofuran

Ts tosyl

### EXAMPLE 1

### Modified Release Composition of Licarbazepine Based upon a HPMC Matrix

A pre-mix is prepared which contains licarbazepine, cellulose microcrystalline and cellulose HPM 603. Purified water is added to the pre-mix which is granulated using a high-shear mixer (Collette 25). The resulting granulation is screened using a Quadracomill then dried using a fluid bed dryer (Aeromatic Fielder MP1). Polyvinyl Polypyrrolidone XL, cellulose microcrystalline, Methocel 60HG 4000 CP and Aerosil 200 are screened with the dried granulation using a Frewitt mill equipped with 1 mm mesh, then mixed using a bin blender (Turbula). Magnesium stearate, is screened through a hand screen (0.8 mm mesh) and added. The final blend is mixed using a bin blender (Turbula).

The final blend is compressed using a Korsch PH250 tabletting press. The tablets are ovaloid, curved 18.0 mm long, 7.1 mm wide and without a breaking bar. The weight of the tablet is 730.00 mg.

Formulation

| Tablet components: | (mg) |
|---|---|
| Licarbazepine | 500.00 |
| Cellulose microcrystalline | 64.30 |
| Cellulose HPM 603 | 14.00 |
| Cellulose microcrystalline | 44.00 |
| Methocel 60 HG 4000 CP | 80.00 |
| Polyvinyl-polypyrrolidone XL | 18.00 |
| Aerosil 200 | 2.50 |
| Magnesium stearate | 7.20 |
| Tablet weight | 730.00 |

### EXAMPLE 2

### Procedure for the Enantioselective Transfer Hydrogenation of 10-Oxo-10,11-dihydro-dibenzo[b,f]azepine-5-carboxylic acid amide to R(−)-10,11-Dihydro-10-hydroxy-5H-dibenz[b,f]azepine-5-carboxamide

To a mixture of 10-oxo-10,11-dihydro-dibenzo[b,f]azepine-5-carboxylic acid amide (300 mg, 1.189 mmol) and RuCl[(1R,2R)-p-TsNCH(C₆H₅)CH(C₆H₅)NH₂](η⁶-p-cymene, Aldrich, Switzerland) (8.8 mg, 0.0138 mmol) in CH₂Cl₂ (15 ml) is added dropwise a premixed solution of formic acid and NEt₃ (5:2, 328 mg:289 mg) at 23° C. and stirred for 10 min. The clear solution is heated to reflux for 16 h. The reaction mixture is cooled to RT, diluted with CH₂Cl₂ (20 ml) and neutralised with aqu. NaHCO₃. After washing with brine the solution is concentrated under reduced pressure. The residue is purified by flash chromatography on silica gel using a 6:1 EtOAc-MeOH mixture as eluent to afford of R(−)-10,11-dihydro-10-hydroxy-5H-dibenzo[b,f]azepine-5-carboxamide (enantiomeric purity (ee) >99% determined by HPLC on Chiracel OD, Retention time: 9.46 min. [α]_{D}^{rt}=−195.3° (ethanol). ¹H-NMR (400 MHz, CDCl₃): 7.70-7.20 (m, 8 H), 5.30 (br s, 1 H), 5.10-4.60 (br s, 2 H), 3.75-3.40 (m, 1 H), 3.20-2.90 (m, 1 H), 2.50 (br s, 2 H). NMR-Datas refer to Lit.: Benes, J et al., J. Med. Chem. 1999, 42, 2582-2587. Molecular weight: 254.291

### EXAMPLE 3

### Procedure for the Enantioselective Transfer Hydrogenation of 10-Oxo-10,11-dihydro-dibenzo[b,f]azepine-5-carboxylic acid amide to S(+)-10,11-Dihydro-10-hydroxy-5H-dibenz[b,f]azepine-5-carboxamide

To a mixture of 10-oxo-10,11-dihydro-dibenzo[b,f]azepine-5-carboxylic acid amide (300 mg, 1.189 mmol) and RuCl[(1S,2S)-p-TsNCH(C₆H₅)CH(C₆H₅)NH₂](η⁶-p-cymene) (11 mg, 0.0173 mmol) in CH₂Cl₂ (15 ml) is added in two portions a premixed solution of formic acid and NEt₃ (5:2, 656 mg:578 mg) at 23° C. and stirred for 10 min. After that formic acid is added (50 μl) and the clear solution is heated to reflux for 16 h. The reaction mixture is cooled to RT, diluted with CH₂Cl₂ (20 ml) and neutralised with aqu. NaHCO₃. After washing with brine the solution is concentrated under reduced pressure. The residue is purified by flash chromatography on silica gel using a 6:1 EtOAc-MeOH mixture as eluent to afford of S(+)-10,11-dihydro-10-hydroxy-5H-dibenzo[b,f]azepine-5-carboxamide (ee >99% by HPLC on Chiracel OD). Retention time: 12.00 min. [α]_{D}^{rt}=+196.6° (ethanol). ¹H-NMR (400 MHz, CDCl₃): 7.70-7.20 (m, 8 H), 5.30 (br s, 1 H), 5.10-4.60 (br s, 2 H), 3.75-3.40 (m, 1 H), 3.20-2.90 (m, 1 H), 2.50 (br s, 2 H). NMR-Datas refer to Lit.: Benes, J et al., J. Med. Chem. 1999, 42, 2582-2587. Molecular weight: 254.291

Alternative production: To a mixture of 10-oxo-10,11-dihydro-dibenzo[b,f]azepine-5-carboxylic acid amide (300 mg, 1.189 mmol) and RuCl[(1S,2S)-p-dansyl-NCH(C₆H₅)CH(C₆H₅)NH₂](η⁶-p-cymene) (8.5 mg, 0.012 mmol) in CH₂Cl₂ (15 ml) is added dropwise a premixed solution of formic acid and NEt₃ (5:2, 328 mg:289 mg) at 23° C. and stirred for 10 min. The clear solution is heated to reflux for 16 h. The reaction mixture is cooled to RT, diluted with CH₂Cl₂ (20 ml) and neutralised with aqu. NaHCO₃. After washing with brine the solution is concentrated under reduced pressure. The residue is purified by flash chromatography on silica gel using a 6:1 EtOAc-MeOH mixture as eluent to afford of S(+)-10,11-dihydro-10-hydroxy-5H-dibenzo[b,f]azepine-5-carboxamide.

### EXAMPLE 4

### Preparation of RuCl[(1S,2S)-p-dansyl-NCH(C₆H₅)CH(C₆H₅)NH₂](η⁶-p-cymene)

a) Preparation of (S,S)-5-dimethylamino-naphthalene-1-sulfonic acid (2-amino-1,2-diphenyl-ethyl)-amide: To a solution of (S,S)-diphenylethylenediamine (250 mg, 1.2 mmol) and triethylamine (0.5 ml) in THF is added dropwise a solution of dansyl chloride (318 mg, 1.2 mmol) in THF (2 ml) at 0° C. After stirring 16 h at RT the solvent is removed in vacuum and the residue is resolved in methylenchloride (20 ml). The organic solution is washed with NaHCO₃ solution (5 ml), dried over Na₂SO₄ and after filtration the solvent is removed. Flash chromatographie afford (S,S)-5-dimethylamino-naphthalene-1-sulfonic acid (2-amino-1,2-diphenyl-ethyl)-amide as yellow oil which crystallizes by drying in vacuum. M: 445.59. ¹H-NMR (400 MHz, CDCl₃): 8.36 (t, J=7.5 Hz, 2 H), 8.17 (dd, J=7.2, 1.2 Hz, 1 H), 7.47 (dd, J=8.8 Hz, 1 H), 7.34 (dd, J=8.5 Hz, 1 H), 7.24-7.16 (m, 4 H), 7.11 (d, J=7.5 Hz, 1 H), 6.99-6.74 (m, 6 H), 4.61 (d, J=8.5 Hz, 1 H), 4.20 (d, J=8.5 Hz, 1 H), 2.80 (s, 6 H)_.

b) Preparation of RuCl[(1S,2S)-p-dansylNCH(C₆H₅)CH(C₆H₅)NH₂(η⁶-p-cymene): A solution of (S,S)-5-dimethylamino-naphthalene-1-sulfonic acid (2-amino-1,2-diphenyl-ethyl)-amide (80 mg, 0.18 mmol), NEt₃ (36 mg, 0.36 mmol) and [RuCl₂(p-cymene)]₂ (55 mg, 0.09 mmol) in 2-propanol is heated at 80° C. for 1 h. The solvent is removed after that und the dark red residue is washed with water (2 ml). The solid is dried in vacuum and used without any purification. M: 715.34.

1. A pharmaceutical oral controlled release composition comprising 10,11-dihydro-10-hydroxy-5H-dibenz[b,f]azepine-5-carboxamide (licarbazepine).

2. A pharmaceutical composition according to claim 1 comprising licarbazepine and a hydrophilic or lipophilic swellable substance.

3. A pharmaceutical composition according to claim 2 wherein a hydrophilic swellable substance is present in a ratio of 5 to 35% by weight of the total composition.

4. A pharmaceutical composition according to claim 1 comprising from 55 to % by weight of licarbazepine of the total composition.

5. A pharmaceutical composition according to claim 1 comprising licarbazepine having a median particle size between 20 and 50 μm.

6. The pharmaceutical composition according to claim 1 consisting of a tablet core and a coating wherein the core comprises licarbazepine optionally, a filler, and at least one hydrophilic swellable substance which is a cellulose ether.

7. The pharmaceutical composition according to claim 1 wherein hydroxypropyl methyl cellulose is employed as the hydrophilic swellable substance.

8. The pharmaceutical composition according to claim 7 wherein the weight ratio of total hydroxypropyl methyl cellulose to licarbazepine is from about 1:3 to about 1:10.

9. A pharmaceutical composition according to claim 1 comprising microcristalline cellulose.

10. A pharmaceutical oral controlled release composition comprising licarbazepine wherein in use 70 to 90% of said licarbazepine is released within 8 to 12 hours in aqueous phosphate buffer having a pH of 6.8 in standard in vitro dissolution tests at 37 degrees Celsius at a stirring rate of 50 rpm for a 500 mg dosage form.

11. A pharmaceutical oral controlled release composition comprising licarbazepine according to claim 10 wherein in use 80 to 90% of said licarbazepine is released within 8 to 12 hours in aqueous phosphate buffer having a pH of 6.8 in standard in vitro dissolution tests at 37 degrees Celsius at a stirring rate of 50 rpm for a 500 mg dosage form.

12. A pharmaceutical oral controlled release composition comprising licarbazepine and a hydrophilic swellable substance adapted to be administered once a day.

13. Use of licarbazepine and excipients as defined in claim 1 for the preparation of a medicament for the treatment of patients with affective disorders.

14. A method of orally administering licarbazepine, e.g., for the treatment of affective disorders, said method comprising orally administering to a patient in need of licarbazepine therapy once-a-day a pharmaceutical composition according to claim 1.
